(19) United States
(12) Patent Application Publication
(10) Pub. No.: US 2020/0206295 A1
(43) Pub. Date: Jul. 2, 2020

(54) Burn Remedy

(71) Applicant: Uzoaku Chukwu, Washington, DC (US)

(72) Inventor: Uzoaku Chukwu, Washington, DC (US)

(21) Appl. No.: 16/723,334

(22) Filed: Dec. 20, 2019

Related U.S. Application Data
(60) Provisional application No. 62/917,768, filed on Dec. 31, 2018.

(51) Int. Cl. A61K 36/889

(52) U.S. Cl. CPC A61K 36/889

(57) ABSTRACT

The use any type of coconut oil immediately on a burn site following a burn incident, reduce and/or resolve the burning pain, and prevent skin blistering. Positive outcome is dependent on time lapse between burn occurrence and coconut oil application. For best result apply any type of coconut oil to the burn site immediately; but will still reduce the burning pain even if time has lapsed and blistering already occurred. A combination of application of cold water first then any coconut oil to a burn site immediately following burn injury reduced healing time.

To reduce the constant burning pain following a burn; also, to prevent blistering of the skin and/or skin loss depending on time of the application of Coconut Oil.

Positive result is dependent on time lapse between burn occurrence and coconut oil application. Coconut Oil must be applied immediately following the burn to achieve the best outcome. Following a burn, rinse the site with cold water, then apply the Coconut Oil. If cold water is not readily available, proceed with application of the Coconut Oil. Coconut Oil can be applied different ways depending on the types of burn injury. The burn remedy can be applied as follows: by hand/fingers; by spray via can/bottle of coconut oil to avoid touching and agitating the burn site to prevent from shifting the skin thereby promote intact skin integrity; with the use of coconut oil infused gauze dressing/wrap; and by using a light brush to apply the coconut oil to avoid adding pressure to the burn site which can cause pain to the injured person.

1. Treatment of burn consists of application of any type of coconut oil to the burn site.

2. Reduction and/or resolution of the burning pain from a burn consists of application of any type of coconut oil to the burn site.

3. Reduction of skin blistering from a burn consist of immediate application of any type of coconut oil to the burn site.
